(19) United States
(12) Patent Application Publication
(10) Pub. No.: US 2024/0341592 A1
(43) Pub. Date: Oct. 17, 2024

(54) RETINAL IMAGING

(71) Applicant: ams International AG, Jona (CH)

(72) Inventors: Volker Zagolla, Eindhoven (NL); Ioannis Papadopoulos, Eindhoven (NL)

(21) Appl. No.: 18/703,725

(22) PCT Filed: Oct. 20, 2022
(86) PCT No.: PCT/EP2022/079242

§ 371 Date: Apr. 23, 2024

(30) Foreign Application Priority Data
Nov. 5, 2021 (EP) 21386067.9

(51) Int. Cl. A61B 3/113; A61B 3/00

(52) U.S. Cl. CPC A61B 3/113; A61B 3/0008

(57) ABSTRACT

A method of imaging a retina an eye includes determining a position of an eye, measuring light reflected or emitted from a point on a retina of the eye, determining a location of the point on the retina based on the position of the eye. The method further includes repeating the steps of determining and measuring over time to provide multiple measurements of light reflected from points in different locations on the retina, and combining the measurements to form an image of the retina.

### CROSS-REFERENCE TO RELATED APPLICATIONS

The present application is a U.S. National Stage Application of International Application PCT/EP2022/079242, filed Oct. 20, 2022, and claims the priority of the European patent application EP 21386067.9 filed Nov. 5, 2021; the entire disclosures of the above-listed applications are hereby explicitly incorporated by reference.

### FIELD

The present disclosure relates to retinal imaging and in particular to point based imaging using the natural rotations of the eye over time.

### BACKGROUND

Retinal imaging (the acquisition of images of the retinal structure of users) can be used in multiple applications such as biometrics, digital biomarkers etc. While different methods of retinal imaging exist there is a continued need for new and improved methods to be developed.

### SUMMARY

According to a first aspect of the present disclosure there is provided a method of imaging a retina of an eye. The method comprises determining a position of the eye (typically the rotational position of the eye but may also be a linear position/displacement depending on the application), measuring light reflected or emitted from a point on a retina of the eye, and determining a location of the point on the retina based on the position of the eye. The method further comprises repeating the steps of determining and measuring over time to provide multiple measurements of light reflected from points in different locations on the retina, and combining the measurements to form an image of the retina.

Embodiments of the disclosure can provide the integration of a single data-point sensing module consisting of a low power light source and a sensing mechanism, e.g. a photodiode, in combination with a functional high-speed eye tracking unit to retrieve and construct a retinal image integrated into a head worn device for continuous measurements.

The step of determining the position may comprise using an eye-tracking unit. Any suitable eye-tracking unit may be used, but a high accuracy and high repetition rate (e.g. >60 Hz) can be advantageous for the disclosed method. By using separate units for eye-tracking and retinal imaging, the system can be made less complex. The eye-tracking is typically used for other purposes, other than retinal imaging, as well.

The step of measuring may comprise illuminating the retina of the eye, focusing light reflected from the point on the retina onto a detector; and receiving the focused light with the detector. The detector typically comprises a photodiode. The detector does not have to be an imaging detector, since only the reflection from a point on the retina is measured at any one time. Therefore, the disclosed solution allows the retina to be imaged using only a single photodiode. The eye may be illuminated in different ways, for example using environmental light. In an embodiment, light is emitted from an emitter and collimated onto the eye. The cornea of the eye focuses the light to a point on the retina.

Measuring may comprise determining one or more of an intensity, a phase (or optical path length), an auto-fluorescence, and a polarisation. Any one or more of these properties may be used to build different images of the retina.

In addition to measuring reflections from the eye, fluorescence may be used to measure light directly emitted from the eye.

The step of measuring typically comprises using an optical element to direct light emitted/reflected from the eye onto one or more photodiodes. In a particular embodiment, the step of measuring may comprise self-mixing interferometry (SMI) whereby the light is emitted by an emitter and the reflected light is received by the same emitter and the output from or the input to the emitter is measured to determine a phase and/or amplitude of the reflected light. A part of the light emitted by the emitter can be directed to a detector, such as a photodiode to measure the output of the emitter and thereby detect the reflected light from the retina.

The steps of determining the position and measuring the reflected light may be repeated at a repetition rate greater than 60 Hz. The greater the repetition rate, the greater the number of points on the retina that can be measured and added to the image for a given period of time.

According to a second aspect of the present disclosure there is provided an optical device for imaging a retina of an eye, the device being suitable for integrating in a head mounted device (e.g. AR glasses). The optical device comprises an eye-tracking unit configured to determine a position of the eye (typically the an eye-tracking unit is configured to determine the rotational position of the eye), and a measuring unit configured to measure light reflected or emitted from a point on a retina of the eye. The device further comprises a processing unit configured to determine a location of the point on the retina based on the position of the eye, and an imaging unit configured to combine multiple measurements of reflected light to form an image of the retina.

The measuring unit may comprise an emitter for illuminating the retina of the eye, and a detector for receiving the light reflected from the point on the retina. The emitter may comprise a light emitting diode, LED, or laser diode. The emitter may comprise a vertical cavity surface emitting laser, VCSEL, configured to emit light having a wavelength in the range of, for example, 850 nm to 1400 nm. A VCSEL can provide a low power solution. The measuring unit may also comprise an optical element for directing light from the emitter onto the eye such as a collimating lens.

The detector typically comprises a photodiode for measuring an intensity of light incident on the photodiode. The detector may comprise a plurality of photodiodes configured to receive light from different points of the retina simultaneously, in order to increase the rate of imaging. Importantly, an imaging sensor is not required in the proposed solution in order to image the retina, but can be used in place of multiple photodiodes. The detector does not by itself receive any spatial information regarding the retina, which instead is provided by the eye-tracking unit.

According to a third aspect of the present disclosure there is provided a head mounted device comprising one or two optical devices according to the second aspect. The optical device may be integrated in a stem (which extends along the side of the head behind the user's eyes) of the head mounted device. The optical element is configured to at least reflect light having a wavelength substantially equal to the light of the emitter, whilst being substantially transparent to light in the visible spectrum. This allows the optical element be placed directly in front of the user's eye and to direct the IR or NIR light from the emitter onto the eye for the retinal imaging, while allowing visible light to pass through the optical element so that the user can see through the device unhindered.

### BRIEF DESCRIPTION OF THE DRAWINGS

The figures do not necessarily reflect the actual proportions, but are intended to illustrate the principles of the various non-limiting embodiments. In the following text, various non-limiting embodiments are described with reference to the following figures:

1A shows a schematic diagram of a setup for retinal imaging with an optical device according to an embodiment;

FIG. 1B shows a schematic diagram of the setup with the eye in a different rotational position;

FIG. 2A shows a schematic diagram of another setup for retinal imaging with an optical device according to another embodiment;

FIG. 2B shows a schematic diagram of the setup with the eye in a different rotational position;

FIG. 3 shows a schematic diagram of a head mounted device according to an embodiment with an optical device for retinal imaging;

FIG. 4 shows a schematic diagram of a head mounted device according to another embodiment with an optical device located in the stem of the device;

FIG. 5 shows a schematic diagram of an optical device according to an embodiment comprising two detectors; and

FIG. 6 shows a schematic of a retinal image formed from the random gaze trajectory of a user.

### DETAILED DESCRIPTION

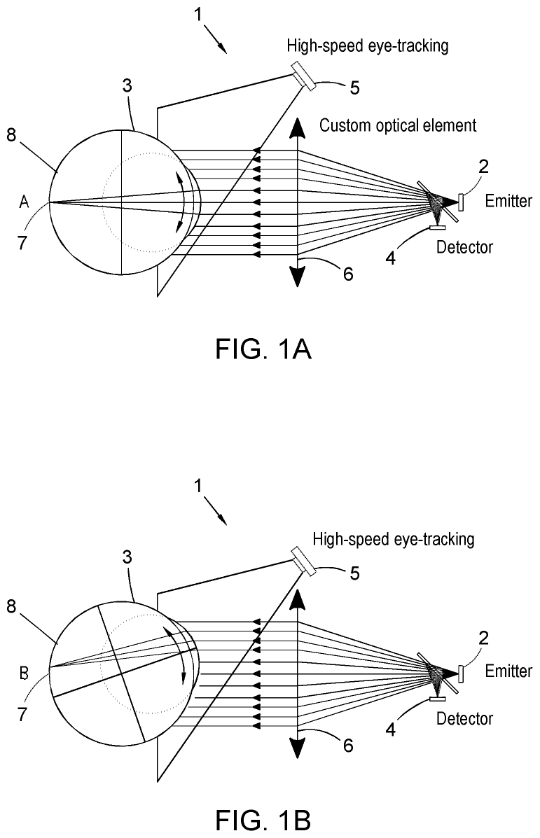

FIG. 1A shows a schematic diagram of an optical device 1 for retinal imaging according to an embodiment. The device 1 comprises an emitter 2 (e.g. comprising one or more VCSELs) for emitting light towards an eye 3 and a detector 4 for detecting light reflected from the eye 3. The device 1 further comprises a separate eye-tracking unit 5 for tracking the rotational position of the eye 3 over time. The optical device 1 also comprises an optical element 6 for directing the light from the emitter onto the eye 3, and to direct reflected light from the eye 3 to the detector 4. The eye 3 focuses at least a part of the incident light to a spot 7 on the retina 8 of the eye 3. Light reflected from the spot 7 is received by the detector 4. Hence, the detector 4 can be used to measure the reflected light from the retina 8.

FIG. 1B shows the optical device 1 after the eye 3 has rotated to a new position. As the eye 3 rotates with respect to the device 1, the location of the spot 7 on the retina 8 changes (from A to B in the diagrams). Hence, over time, an image of the retina 8 can be obtained by combining multiple measurements.

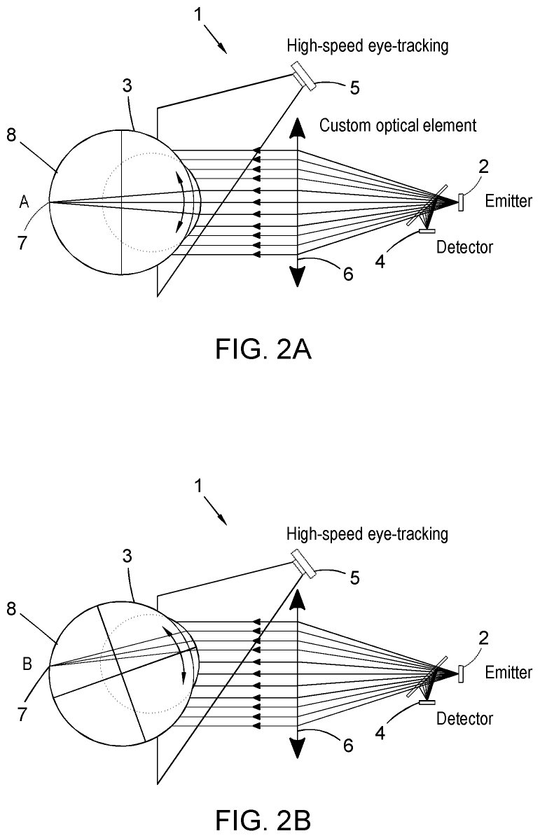

FIGS. 2A and 2B illustrate another embodiment in which self-mixing interferometry (SMI) is used to measure the light reflected from the retina 8. For clarity, the same reference numerals have been used in different figures to denote similar or equivalent features and are not intended to limit the scope of the illustrated embodiments.

FIG. 2A shows a schematic diagram of an optical device 1 for retinal imaging. The device comprises an emitter 2 and a detector 4. The detector 4 is arranged to measure the output of the emitter 2 (instead of the light reflected from the eye 3 as in FIGS. 1A and 1B). A major part (e.g. 99%) of light emitted by the emitter 2 is directed onto the eye 3 by the optical element 6. The eye 3 focuses the light to a spot 7 on the retina 8. Light reflected from the spot 7 is then received back in the emitter 2. The received light interferes with the light in the emitter 2 and thereby changes the output from the emitter 2. This change in output is measured by the detector 4, which receives a small part of the emitted light.

FIG. 2B shows the device 1 when the eye 3 has rotated, so that the location of the spot 7 on the retina 8 has changed accordingly.

In general for the embodiments, the rotational position of the eye 3 can be mapped to the location of the focus spot 7 on the retina 8. Multiple measurements can be combined to provide an image of the retina 8. The image may plot the intensity of reflected light from the retina 8 against the location on the retina 8.

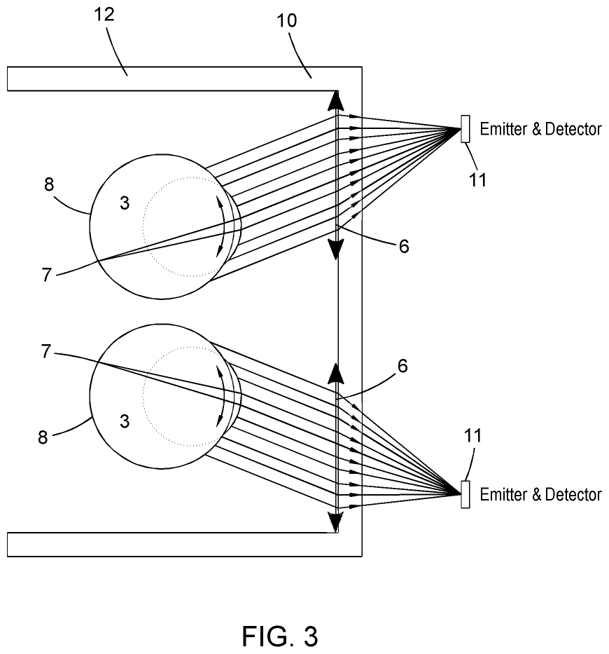

FIG. 3 shows a system 9 with two optical devices 1 integrated in a pair of glasses 10 (e.g. AR smart glasses). The glasses 10 are a head mounted device, which holds the optical device 1 fixed relative to the eye 3. This allows the optical device 1 to measure the retinal reflection over the time that the glasses 10 are worn, in order to provide a sufficiently detailed image of the retina 8. The optical devices 1 each comprises an emitter and detector apparatus 11 for emitting light and for receiving reflected light from a respective eye 3. The emitter and detector apparatus 11 may use SMI, e.g. using a VCSEL emitter. The emitter and detector apparatus 11 may comprise an emitter 2 and detector 4 as shown in e.g. FIG. 1A or FIG. 2A.

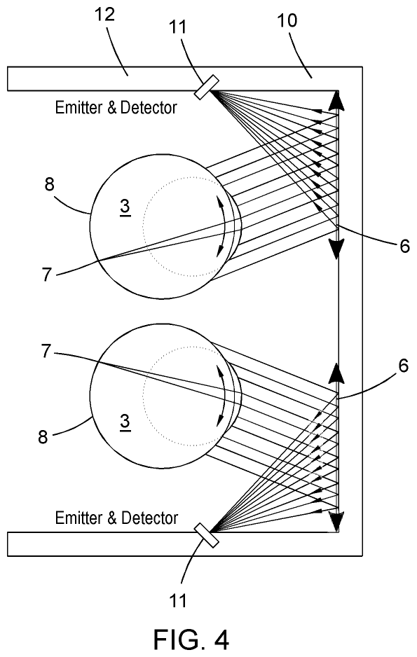

FIG. 4 shows a schematic diagram of a system according to an embodiment similar to that of FIG. 3, but wherein the emitter and detector apparatus 11 is located in the stem 12 of the glasses 10. The optical element 6 now also reflects the light towards the eyes 3. The embodiment can provide a more compact solution.

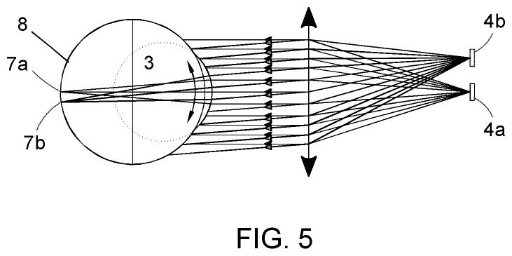

FIG. 5 shows a schematic diagram of an optical device 1 according to an embodiment comprising two detectors 4a and 4b (or two photodiodes of the same detector). Multiple adjacent detectors 4a and 4b located in the image plane can be arranged to image different locations 7a and 7b on the retina 8. This adds additional data points at any given time, at the expense of other system parameters, e.g. power consumption.

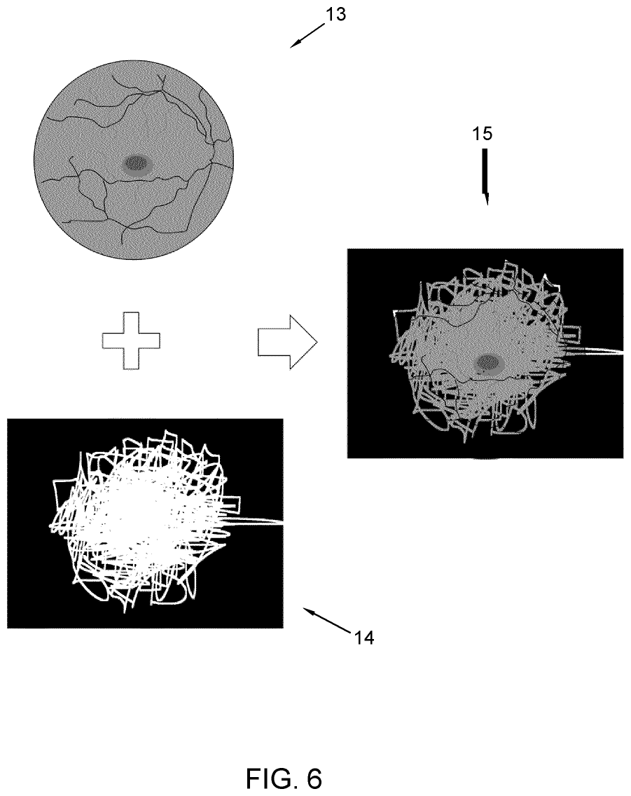

FIG. 6 shows a schematic image of a retina 13 and the trajectory of a user's gaze over time 14. The combination 15 of the image 13 and the trajectory 14 shows a retinal image as may be provided by an embodiment.

Embodiments allow a single data point of the retina to be measured by measuring the reflection from the retina with the detector. The information gathered from this point is then related to the current eye position via the eye tracking unit. The combination of this data allows the construction of an image of the retina over time, relying on the continuous, natural movement of the eye, since each eye position will return a specific data point. The integration into a head worn device allows for the continuous measurement over time.

The use of the passive movement of the eye and a single data point in order to construct a retinal image can provide the following advantages:
Very power-efficient architecture with a minimum number of components.
Low complexity due to a small number of components.
Does not require mechanical or moving elements, as the solution relies on the natural eye movement.

Although specific embodiments have been described above, the claims are not limited to those embodiments. Each feature disclosed may be incorporated in any of the described embodiments, alone or in an appropriate combination with other features disclosed herein.

Reference Numerals

| | |
|---|---|
| 1 | Optical device |
| 2 | Emitter |
| 3 | Eye |
| 4 | Detector |
| 5 | Eye-tracking unit |
| 6 | Optical element |
| 7 | Spot |
| 8 | Retina |
| 9 | Optical system |
| 10 | Glasses |
| 11 | Emitter and detector apparatus |
| 12 | Stem |
| 13 | Retinal image |
| 14 | Eye gaze trajectory |
| 15 | Combined image |

1. A method of imaging a retina of an eye, the method comprising:
determining a position of the eye;
measuring light reflected or emitted from a point on the retina of the eye;
determining a location of the point on the retina based on the position of the eye;
repeating the steps of determining and measuring over time to provide multiple measurements of light reflected from points in different locations on the retina; and
combining the measurements to form an image of the retina.

2. The method according to claim 1, wherein the step of determining the position comprises using an eye-tracking unit.

3. The method according to claim 1, wherein measuring comprises:
illuminating the retina of the eye; and
focusing light reflected from the point on the retina onto a detector; and
receiving the focused light with the detector.

4. The method according to claim 3, wherein the step of illuminating comprises emitting light with an emitter and directing the emitted light onto the eye with an optical element.

5. The method according to claim 1, wherein the detector comprises a photodiode.

6. The method according to claim 1, wherein measuring comprises determining one or more of an intensity, a phase, an auto-fluorescence, and a polarisation.

7. The method according to claim 1, wherein measuring comprises self-mixing interferometry, SMI, so that the light is emitted by an emitter and the reflected light is received by the same emitter and the output from or the input to the emitter is measured to determine a phase and/or amplitude of the reflected light.

8. The method according to claim 7, wherein a part of the light emitted by the emitter is directed to a detector.

9. The method according to claim 1, wherein determining the position and measuring the reflected light are repeated at a repetition rate greater than 60 Hz.

10. An optical device for imaging a retina of an eye, the device being suitable for integrating in a head mounted device, the optical device comprising:
an eye-tracking unit configured to determine a position of an eye;
a measuring unit configured to measure light reflected or emitted from a point on a retina of the eye;
a processing unit configured to determine a location of the point on the retina based on the position of the eye; and
an imaging unit configured to combine multiple measurements of reflected light to form an image of the retina.

11. The optical device according to claim 10, wherein the measuring unit comprises
an emitter for illuminating the retina of the eye; and
a detector for receiving the light reflected from the point on the retina.

12. The optical device according to claim 11, wherein the emitter comprises a light emitting diode, LED, or laser diode.

13. The optical device according to claim 11, wherein the emitter comprises a vertical cavity surface emitting laser, VCSEL, configured to emit light having a wavelength in the range of 800 nm to 1400 nm.

14. The optical device according to claim 11, wherein the measuring unit further comprises an optical element for directing light from the emitter onto the eye.

15. The optical device according to claim 10, wherein the detector comprises a photodiode for measuring an intensity of light incident on the photodiode.

16. A head mounted device comprising one or two optical devices as claimed in claim 10.

17. The head mounted device according to claim 16, wherein the or each optical device is integrated in a stem of the head mounted device and the optical element is configured to at least reflect light having a wavelength substantially equal to the light of the emitter, whilst being substantially transparent to light in the visible spectrum.
